(19) United States
(12) Patent Application Publication
(10) Pub. No.: US 2012/0209129 A1
(43) Pub. Date: Aug. 16, 2012

(54) Blood pressure monitoring cuff with acoustic sensor

(76) Inventors: Robert M. Smith, Hartford, CT (US); Michael Jorgensen, Ashland, MA (US)

(21) Appl. No.: 13/374,553

(22) Filed: Jan. 3, 2012

Related U.S. Application Data
(60) Provisional application No. 61/460,601, filed on Jan. 4, 2011.

(51) Int. Cl. A61B 5/0225

(52) U.S. Cl. 600/493

(57) ABSTRACT

A system and method for a BP monitoring cuff that has an integrated acoustic sensor used to acquire Korotkoff sounds during partial occlusion of the arterial vessel.

### CROSS REFERENCE TO RELATED APPLICATIONS

This application claims the benefit of U.S. Provisional Application No. 61/460,601, filed on Jan. 4, 2011, the disclosure which is incorporated herein by reference in its entirety.

### BACKGROUND OF THE INVENTION

The invention relates generally to Non-Invasive Blood Pressure (NIBP) monitoring. More specifically, the invention relates to a Blood Pressure (BP) monitoring cuff that has an integrated acoustic sensor that is used to acquire Korotkoff sounds during arterial vessel partial occlusion.

When the human heart contracts, a pressure gradient is generated to move blood through the systemic and pulmonary arteries of the cardiovascular system. The contractile phase of the cardiac cycle is called systole, and the relaxation phase is diastole. The volume of blood flowing through the arteries exerts a pressure on their internal walls, which can be palpated on the skin in several locations.

Blood Pressure (BP) is a measurement of the systolic and diastolic pressures of the cardiac cycle and provides valuable diagnostic information for clinical professionals. BP may be measured non-invasively using the auscultatory or oscillometric methods.

The auscultatory method uses a stethoscope and an occluding cuff. The cuff is placed around the upper arm approximately at the same vertical height as the heart, attached to a manometer. A cuff of appropriate size is fitted, and then inflated manually by repeatedly squeezing a rubber bulb until the artery is completely occluded. The stethoscope is used to listen to the brachial artery at the cubital fossa.

The cuff pressure is slowly released at a rate of approximately one to five mmHg per second, and when the cuff pressure reaches systolic blood pressure, blood begins to flow' in the artery and the turbulent flow creates a whooshing sound, an indication of the first Korotkoff sound. The pressure at which this sound is first heard is the systolic BP. The cuff pressure is further released until no sound can be heard, an indication of the fifth Korotkoff sound and diastolic BP. The auscultatory method is the predominant method of clinical measurement due to its accuracy, reliability, and non-invasive nature.

There are five Korotkoff sounds: 1) the snapping sound first heard at the systolic pressure. Clear tapping, repetitive sounds for at least two consecutive beats is considered the systolic pressure, 2) the murmurs heard for most of the area between the systolic and diastolic pressures, 3) a loud, crisp tapping sound, 4) at pressures within 10 mmHg above the diastolic blood pressure, thumping and muting, and 5) silence as the cuff pressure drops below the diastolic pressure. The disappearance of sound is considered diastolic blood pressure, two mmHg above the last sound heard. The second and third Korotkoff sounds have little clinical significance.

The oscillometric method of measuring BP uses the pressure oscillations produced by the pulses of the brachial artery. An electronic version of the method uses a cuff with automatic inflation and deflation and an electronic pressure sensor to observe cuff pressure oscillations. Electronics automatically interpret the cuff pressure oscillations and display BP.

The cuff is inflated to a pressure initially in excess of the systolic arterial pressure and then reduced to below diastolic pressure. When blood flow is occluded or unimpeded, cuff pressure is constant. When blood flow is present, but restricted, the cuff pressure which is monitored by the pressure sensor varies with the cyclic expansion and contraction of the brachial artery and oscillates. The values of systolic and diastolic pressure are calculated using an algorithm and are displayed. To maintain accuracy, the pressure sensor requires periodic calibration.

The different methods do not give identical results. An algorithm and experimentally obtained coefficients are used to adjust the oscillometric method to arrive at BP measurements that match the auscultatory method. Oscillometric monitors may produce inaccurate readings in patients with heart and circulation problems since the BP is not measured from raw data and may include intrinsic errors from their algorithms, pressure sensor, subject rhythm disturbances, variation in a patient's compliance or patient movement during measurement. Since many oscillometric devices have not been validated, caution must be given as many may not be suitable in clinical and acute care settings.

The auscultatory method is less dependent on empiric algorithms for the determination of systolic and diastolic values. However, the acoustic sensor must be accurate, positioned correctly over the brachial artery and isolated from noise.

Accurate blood pressure readings are important since inaccuracies can lead to clinical misdiagnosis. Hypertensive patients are frequently prescribed medication to lower their blood pressure, which may require taking medication for an extended period of time. Therefore, the consequences of a misdiagnosis of hypertension may be detrimental to the patient's quality of life.

The Association for the Advancement of Medical Instrumentation (AAMI) published the SP10 national standards for manual, electronic, and automated sphygmomanometers in 2002. All NIBP monitors and blood pressure cuffs must conform to these standards. The document also specifies requirements for transducing elements including pressure transducers, microphones, and oscillometric sensors used in BP monitors. The specifications require that great care should be taken to avoid movement of the stethoscope during the measurement, as the device's microphone or oscillometric sensor (depending on the type of device) can inadvertently sense the movement as noise or Korotkoff sounds.

NIBP monitors that use the oscillometric method are susceptible to patient movement, mechanical vibrations when inflating/deflating the cuff and noise manifest at the cuff that may synchronize with the arterial oscillations and produce errors in BP measurement. NIBP monitors that use the auscultatory method are susceptible to errors caused by misplacing the stethoscope over the brachial artery or applying too little or too great a pressure to the cubital fossa.

What is desired is a blood pressure cuff that can more accurately transduce arterial sounds for use in automatic auscultation NIBP monitors.

### SUMMARY OF THE INVENTION

The inventors have discovered that it would be desirable to have a system and method for a BP monitoring cuff that has an integrated acoustic sensor used to acquire Korotkoff sounds during partial occlusion of the arterial vessel.

One aspect of the invention is a Non-Invasive Blood Pressure (NIBP) monitoring cuff. Cuffs according to this aspect of the invention include a cuff comprising a top surface and a bottom surface, the top and bottom surfaces configured as mirror images of one another and define an interior aperture, an inflation tubing barb, wherein the top and bottom surfaces are of a flexible air-tight material and the top, bottom and tubing barb are coupled together and define an internal bladder, and a fastening means applied to the top and bottom surfaces to secure the cuff when wrapped, and an acoustic sensor, and a suspension, wherein the suspension is configured to be positioned in the interior aperture and coupled to the cuff, and the sensor is configured to be coupled to the suspension, wherein the suspension provides acoustic isolation between the cuff and the acoustic sensor, and limits acoustic sensor movement during bladder inflation and deflation after cuff placement.

Another aspect of the invention provides a Non-Invasive Blood Pressure (NIBP) monitoring method. Methods according to this aspect of the invention include positioning a blood pressure cuff having an inflatable bladder and an acoustic sensor with the acoustic sensor in contact with a surface directly in contact with an artery, selecting a first predetermined cuff bladder deflation rate, inflating the cuff bladder, monitoring acoustic sensor signal values and bladder pressure values during bladder inflation, determining an approximate systolic Blood Pressure (BP), inflating the cuff bladder to a pressure exceeding the approximate systolic BP to occlude the artery, initiating cuff bladder deflation comprising bleeding the cuff bladder pressure at the first predetermined cuff bladder deflation rate, trending the acoustic sensor signal values versus the cuff bladder pressure values starting at bladder deflation initiation, determining a final acoustic sensor signal value from the trend of acoustic sensor signal values versus cuff bladder pressure values, and bleeding the cuff bladder pressure at a second predetermined cuff bladder deflation rate until the cuff bladder is completely deflated, and determining a systolic BP and a diastolic BP from the acoustic sensor signal values versus bladder pressure values trend.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a top view of an exemplary BP monitoring cuff with acoustic sensor.

FIG. 2 is a bottom view of the BP cuff shown in FIG. 1.

FIG. 3 is a section view along line 3-3 in FIG. 1.

FIG. 4A is an exemplary separate inflatable bladder located within the cuff.

FIG. 4B is an exemplary configuration of the suspension.

FIG. 5 is a view of the BP monitoring cuff in use.

FIG. 6 is an exemplary BP processing unit.

FIG. 7 is a BP monitoring method.

FIG. 8 is a graph of acoustic sensor output values in percent (ordinate) versus cuff bladder pressure in mmHg (abscissa).

### DETAILED DESCRIPTION

Embodiments of the invention will be described with reference to the accompanying drawing figures wherein like numbers represent like elements throughout. Before embodiments of the invention are explained in detail, it is to be understood that the invention is not limited in its application to the details of the examples set forth in the following description or illustrated in the figures. The invention is capable of other embodiments and of being practiced or carried out in a variety of applications and in various ways. Also, it is to be understood that the phraseology and terminology used herein is for the purpose of description and should not be regarded as limiting. The use of “including,” “comprising,” or “having,” and variations thereof herein is meant to encompass the items listed thereafter and equivalents thereof as well as additional items.

The terms “mounted,” “connected,” and “coupled,” are used broadly and encompass both direct and indirect mounting, connecting, and coupling. Further, “connected,” and “coupled” are not restricted to physical or mechanical connections or couplings.

It should be noted that the invention is not limited to any particular software language described or that is implied in the figures. One of ordinary skill in the art will understand that a variety of alternative software languages may be used for implementation of the invention. It should also be understood that some of the components and items are illustrated and described as if they were hardware elements, as is common practice within the art. However, one of ordinary skill in the art, and based on a reading of this detailed description, would understand that, in at least one embodiment, components in the method and system may be implemented in software or hardware.

Embodiments of the invention provide methods, system frameworks, and a computer-usable medium storing computer-readable instructions. The invention may be deployed as software as an application program tangibly embodied on a program storage device. The application code for execution can reside on a plurality of different types of computer readable media known to those skilled in the art.

Embodiments provide an inflatable BP monitoring cuff with an integrated acoustic sensor. The sensor is acoustically decoupled from the cuff to obviate acquiring noise manifest during cuff deflation. As the cuff deflates, the sensor suspension maintains a predetermined pressure between the sensor's face and a patient's brachial artery due to the suspension and modulus of elasticity (Young's modulus) of the top and bottom surface materials. The sensor allows for accurate Korotkoff sounds to be acquired. Embodiments also provide an automated controller that comprises a sphygmomanometer or inflation/deflation system in conjunction with an acoustic signal processing unit that displays a BP measurement.

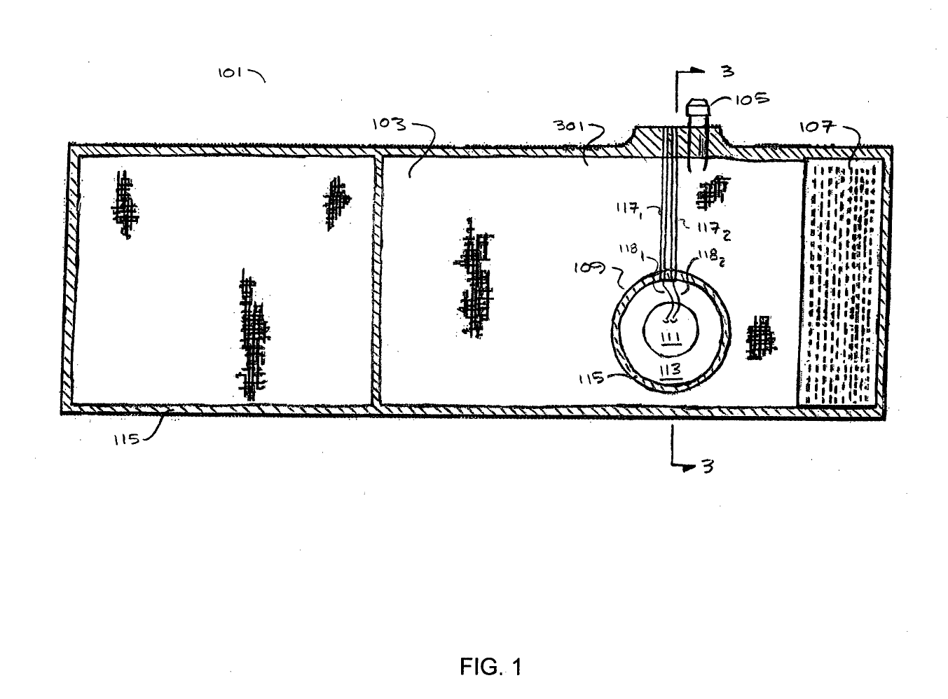

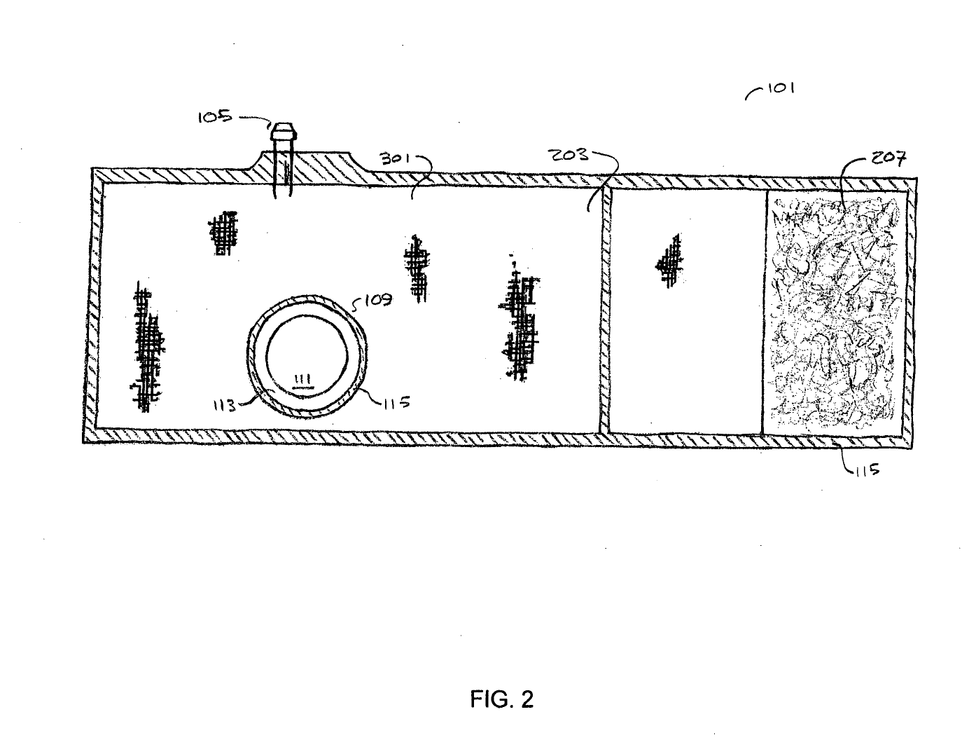

FIG. 1 shows a cuff embodiment 101 top view and FIG. 2 shows the bottom view. The cuff 101 comprises a top surface 103, a bottom surface 203, an inflation tubing barb 105 and a fastening means 107, 207 to secure the cuff when wrapped around a patient's upper arm. The fastening means 107, 207 is positioned in matching correspondence on the top 103 and bottom 203 surfaces and allows for a range of upper arm circumferences. The fastening means 107, 207 may be a hook and loop fastener or other closure system.

The top 103 and bottom 203 surfaces may be configured as mirror images of one another and define an aperture 109 configured to position an acoustic sensor 111. The top 103 and bottom 203 surfaces define a length and width such that the cuff 101 may easily wrap around a patient's bicep to obtain a blood pressure measurement. The cuff 101 may have indicia (not shown) describing proper positioning and usage. The indicia may be applied lettering, for example, by silk screening, or a surface feature of the cuff 101, for example, stitching or embossing.

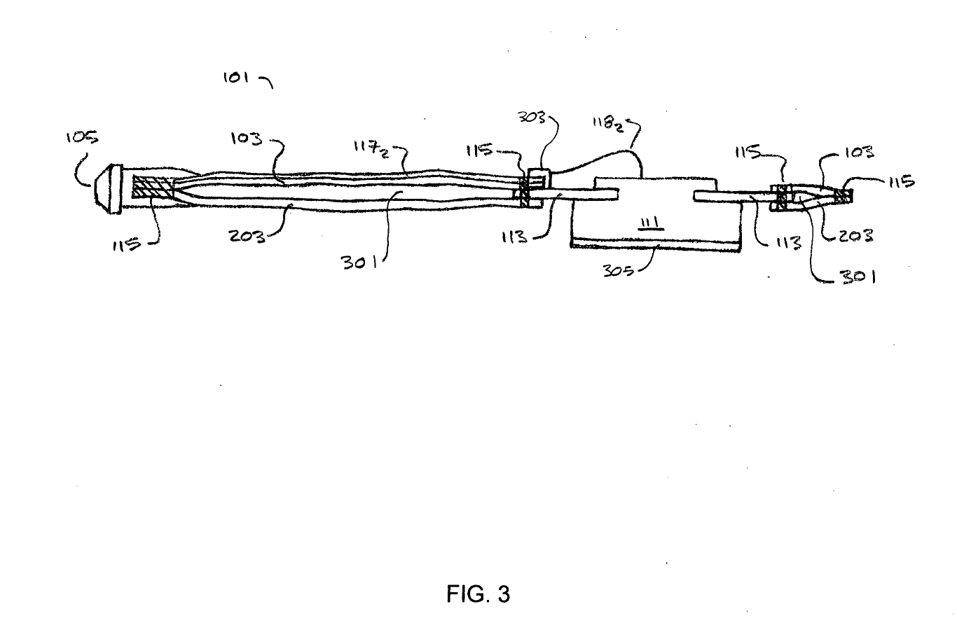

The acoustic sensor 111 is decoupled from the top 103 and bottom 203 surfaces using a suspension 113. FIG. 3 shows a section view of the cuff 101.

The top 103 and bottom 203 cuff surfaces define a partial internal bladder 301 and are of a flexible air-tight material. The top surface 103 material may be different from the bottom surface 203 material to define bladder 301 inflation/deflation properties. As shown in FIG. 3, the top 103, bottom 203, tubing barb 105 and suspension 113 are sandwiched together and hermetically sealed 115. The seal 115 may be accomplished by RF, ultrasonic, friction welding, or other methods known in the art depending on the top 103 and bottom 203 materials and suspension 113 material, and defines the bladder 301 portion of the cuff 101.

The bottom 203 surface is in direct contact with the patient and may be formed of a material that is resistant to absorption of biofluids and is easily cleaned and sanitized between uses. A suitable material may be a polymer or copolymer, such as nylon or another biocompatible, impermeable elastomeric material. The material may be coated with a biocompatible coating layer such as polyurethane to provide moisture resistance. In certain embodiments, a disposable cover may be positioned over the cuff 101 to provide a more sanitary means of usage between patients.

The cuff 101 is intended for repeated usage, but may be disposable in other embodiments.

The top 103 surface material may be formed of a similar material or a different material.

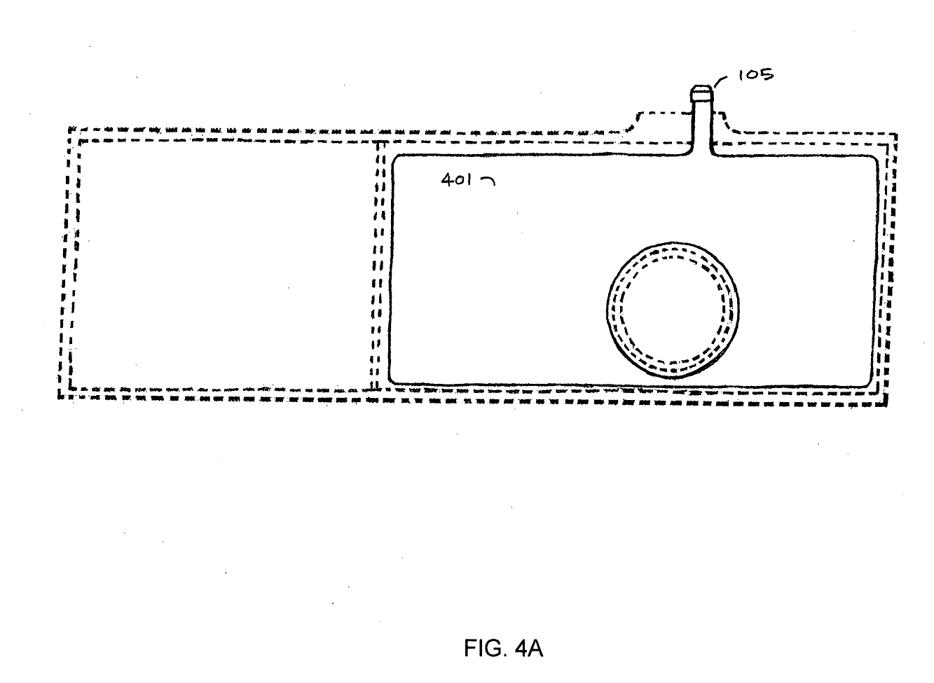

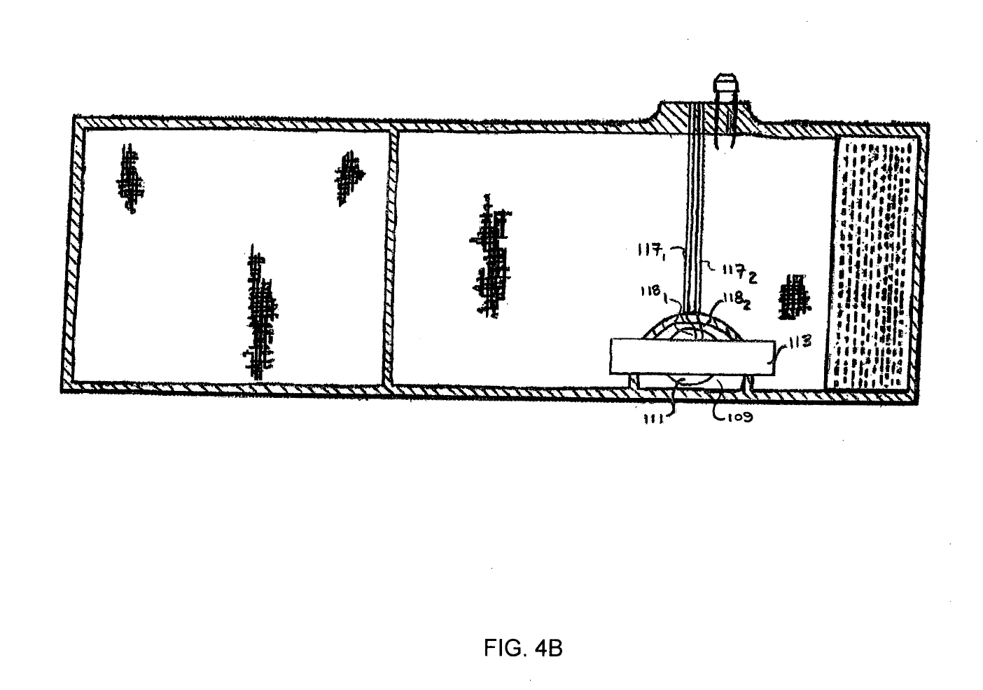

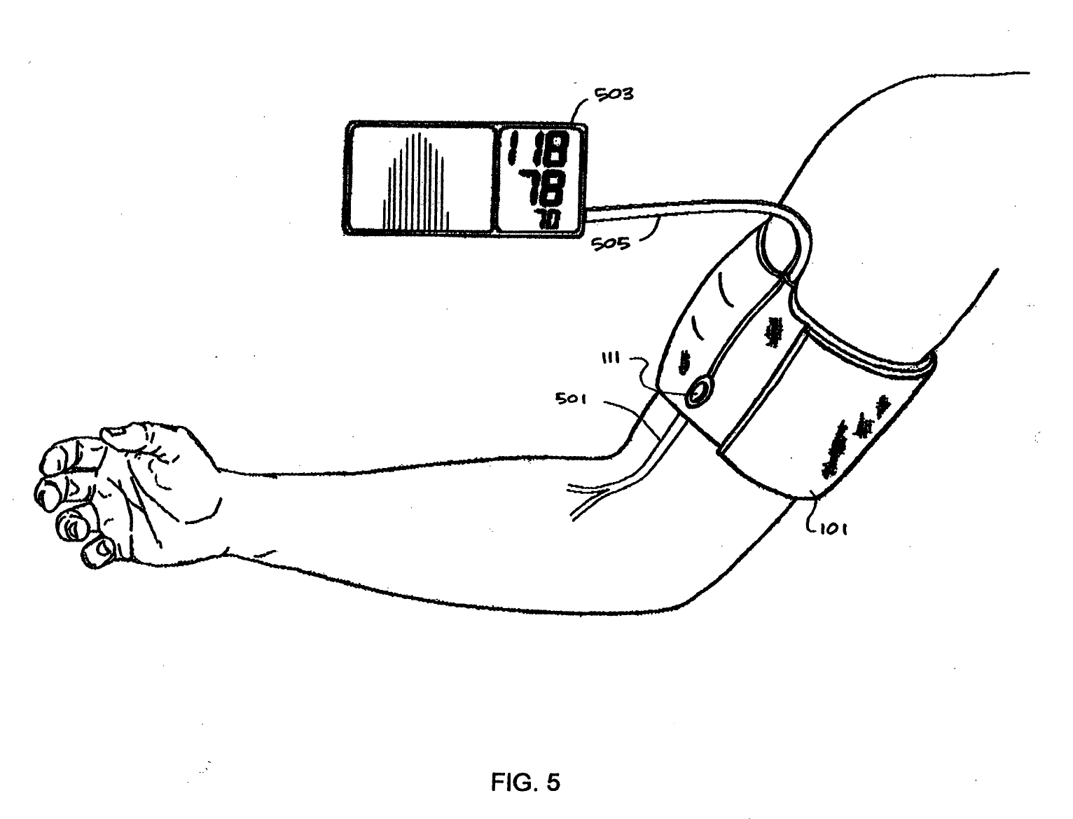

The connection of the top 103 and bottom 203 surfaces to form an inflatable bladder is not a limited means for inflation and deflation. FIG. 4A shows a separate inflatable bladder 401 located within the top 103 and bottom 203 surfaces. FIG. 4B shows an alternate (rectangular) configuration of the suspension 113.

The suspension 113 material provides acoustic isolation between the top 103 and bottom 203 surfaces and the acoustic sensor 111, and limits vertical and horizontal movement during bladder 301 pressurization after cuff 101 placement. The suspension 113 provides sufficient rigidity to hold the sensor 111. For this embodiment, the suspension 113 is circularly shaped and has an aperture in the center configured to allow a flange of the sensor 111 to pass through. Materials to form the suspension 113 may include but are not limited to woven polyester and rubber, woven elastic fabrics, formed elastics or latex-free elastics. The suspension 113 may also be a corrugated fabric disk. The sensor 111 may be bonded to the suspension 113.

Cuff 101 inflation and controlled deflation is performed using the tubing barb 105 coupled via a flexible tube to a rubber bulb or automatic inflation/deflation pump (not shown). Two or more conductors 117₁, 117₂ (collectively 117) may be integrated with the top surface 103 using, for example, flexible plastic conductors, for coupling the acoustic sensor 111 with an outboard signal processing unit 503. The number of conductors 117 is determined by the type of acoustic sensor 111 used and each end of the conductor 117 may be reinforced with a stiffener to provide strain relief and aid coupling. To further decouple the sensor 111 from the cuff 101, flexible wires 118₁, 118₂ (collectively 118) complete the circuit to and from the sensor 111 via a coupler 303.

The inflatable bladder 301 is capable of inflating to pressures that are necessary to collapse the brachial artery to obtain a BP measurement. The cuff 101 width is optimally 0.40 times the circumference of the arm at the midpoint of the intended range of the cuff. The length of the cuff bladder 301 should be approximately 0.80 times the circumference of the arm at the midpoint of the intended range of the cuff. For this reason, the blood pressure cuff 101 may be manufactured in more than one size to accommodate children, adults and obese patients.

The sensor 111 may comprise transducers, microphones, ceramic bimorphs, piezoelectric materials or other sensor types known in the art. Korotkoff sounds exist in a frequency range of 18 to 250 Hz, and arterial pulsations can exist at frequencies as low as 1 Hz. Therefore, the sensor 111 may acquire frequencies between 1 to 250 Hz or more. The detection face 305 of the sensor 111 may be curved.

FIG. 5 shows the cuff 101 in use. When wrapped around a patient's upper arm, the acoustic sensor 111 detection face 305 is placed directly over the brachial artery 501. The suspension 113 exerts a predetermined amount of force to the sensor 111 to maintain uniform pressure on the cubital fossa during bladder 301 inflation and deflation. The application of constant pressure provides accurate BP measurements by transducing a consistent signal from the brachial artery 501 to a cuff inflation/deflation/signal processing unit 503. Variance in sensor 111 pressure against the patient's skin can result in inconsistent and inaccurate BP data. A flexible tube with integrated conductors 505 couples the cuff 101 to the processing unit 503.

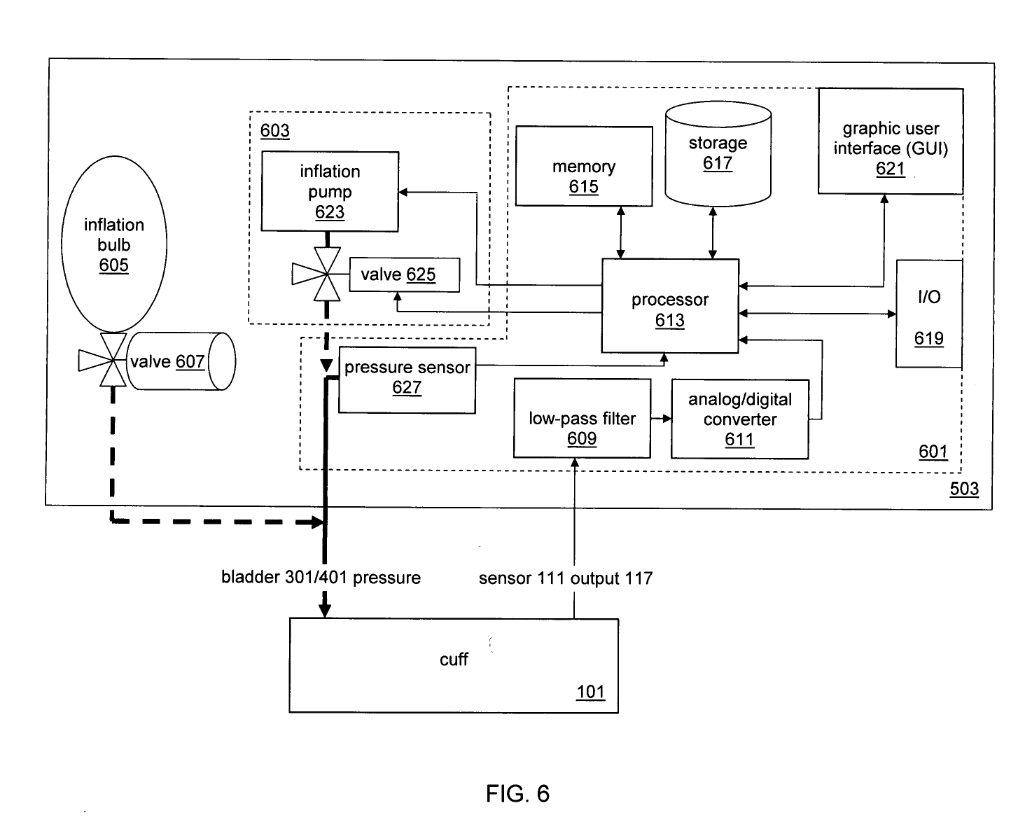

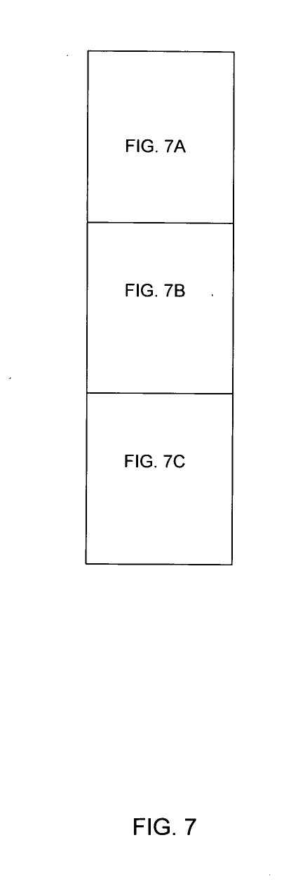

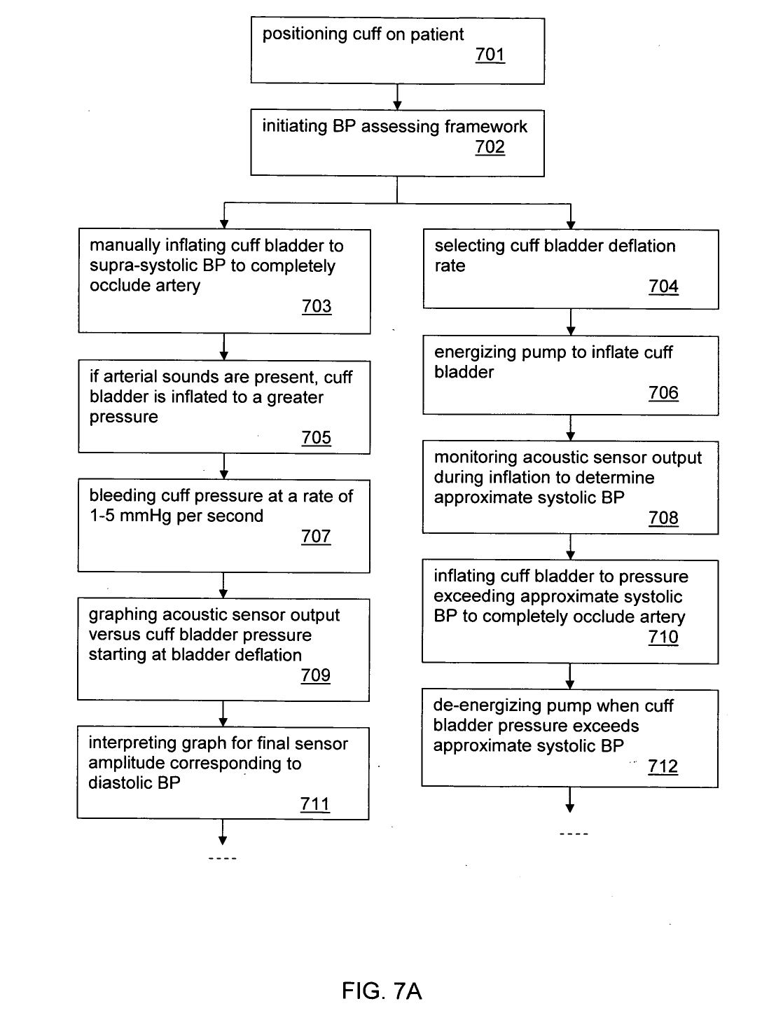

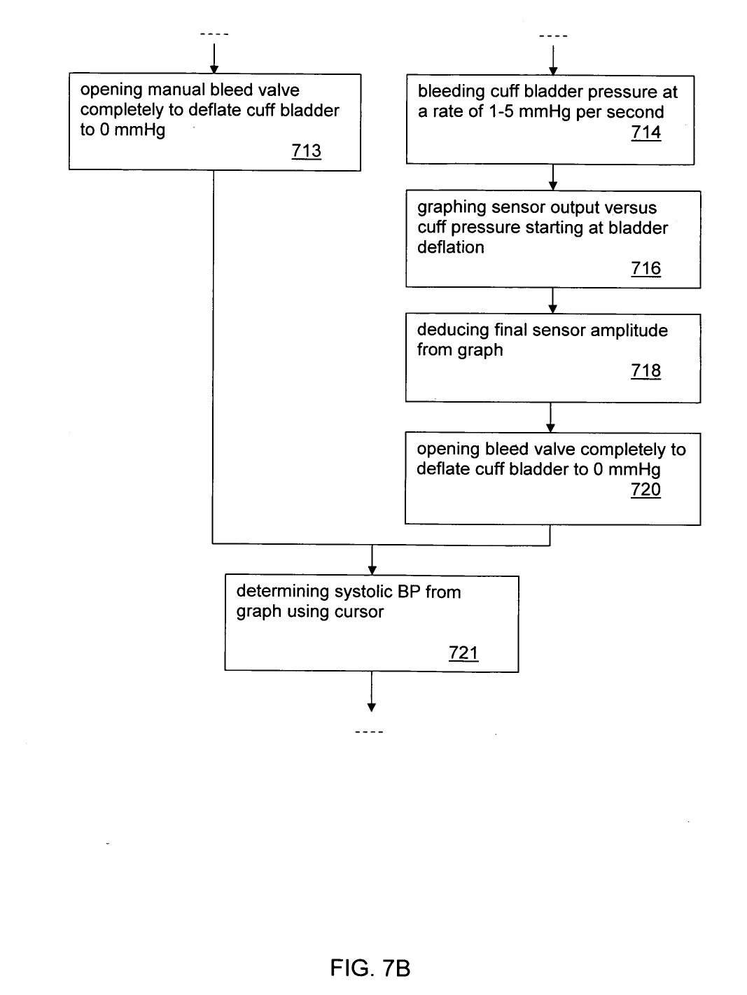

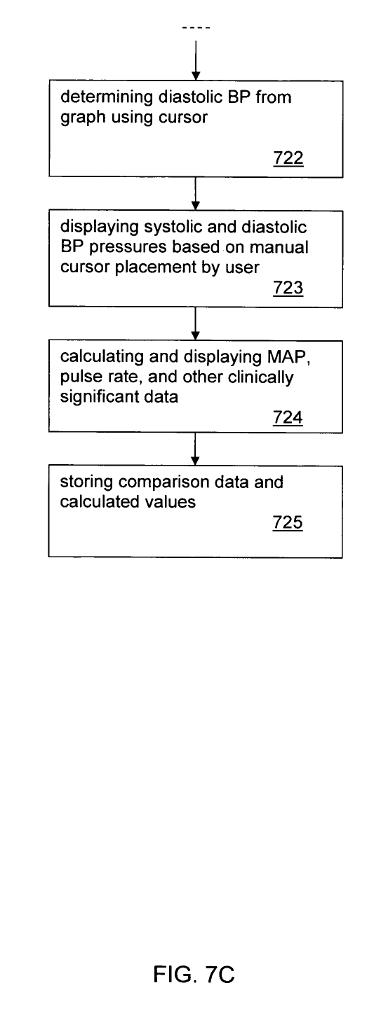

FIG. 6 shows embodiments of the processing unit 503 and FIG. 7 shows a method. The processing unit 503 calculates and displays values for systole, diastole, mean arterial pressure (MAP), pulse rate and other clinical data, and comprises a BP assessing framework 601 and either an automatic inflation/deflation system 603 or a manual inflation (squeeze) bulb 605 (with bleed valve 607) to inflate and deflate the cuff 101 bladder 301/401.

The BP assessing framework 601 comprises a Low Pass Filter (LPF) 609, an Analog/Digital Converter (ADC) 611, a processor 613, memory 615, a data store 617, I/O 619, a Graphic User Interface (GUI) 621 and a pressure sensor 627. The processor 613 receives cuff 101 bladder 301/401 pressure measurements from the pressure sensor 627. The GUI 621 provides a configurable display and allows a user to input commands. The GUI 621 may be a multi-touch screen employing gesture-touch.

The LPF 609 receives arterial sound signals from the acoustic sensor 111 and in conjunction with the ADC 611. The ADC 611 provides an adequate sampling rate based on the sensor 111 signal bandwidth and Nyquist-Shannon theorem,

fₛ≧2B, (1)

where fₛ is the required sampling frequency, B is the maximum sensor 111 bandwidth and 2 is the multiplier required by the Nyquist-Shannon theorem. The discrete time output of the ADC 611 is input to the processor 613.

The processor 613 timestamps the output of the ADC 611 (sensor 111) to provide real-time data logging, for example, sensor 111 output versus cuff pressure data over an indefinite period that allows for multiple inflations/deflations. Results and acquired data are stored in the data storage 617 and may be uploaded to a server/processor (not shown) via I/O 619 for additional data analysis.

Depending on the embodiment, the processor 613 may be coupled to the automatic inflation/deflation system 603 which comprises a pump 623 and a bleed valve 625. The pump 623 and valve 625 receive control signals from the processor 613 according to the program.

Referring now to FIG. 7, using indicia on the cuff 101, the cuff 101 is wrapped around a patient's upper arm with the acoustic sensor 111 positioned above the brachial artery 501 (step 701). The assessing framework 601 is initiated (step 702).

In one embodiment, the cuff 101 may be automatically inflated. The user accesses a menu via the GUI 621 and selects a cuff 101 bladder 301/401 deflation rate in a range of 1-5 mmHg per second (step 704). The inflation pump 623 is energized and the cuff 101 bladder 301/401 begins to inflate (step 706). During cuff bladder inflation, the arterial sound 111 magnitudes and cuff bladder pressure 627 are monitored and displayed on the GUI 621, and compared with each other to determine an approximate systolic BP (step 708). Alternatively, the inflation pump 623 may inflate initially to a high pressure, for example, 180 mmHg. If arterial sounds manifest immediately when cuff 101 bladder 301/401 deflation begins, the cuff 101 bladder 301/401 is inflated to a pressure greater than 180 mmHg, for example, 200 mmHg.

At cuff 101 bladder 301/401 pressures less than diastole, the brachial artery is not occluded and no acoustic sounds are manifest. However, at partial occlusion, acoustic sounds begin to manifest themselves.

Once an approximate systolic BP measurement is made, the inflation pump 623 inflates the cuff 101 bladder 301/401 to a pressure greater than the approximate systolic BP (step 710). At this pressure, the brachial artery is completely occluded resulting in no blood flow. The inflation pump 623 is then de-energized to allow for bladder 301/401 deflation (step 712).

The cuff 101 bladder 301/401 pressure 627 begins to fall at the previously chosen rate by controlling the bleed valve 625 (step 714). The acoustic sensor 111 acquires sound manifesting in the brachial artery (during partial occlusion), and outputs a voltage magnitude that is compared with the bladder 301/401 pressure 627. A comparison of acoustic sensor 111 sound magnitude versus bladder pressure 627 is captured and stored 617 while the cuff 101 bladder 301/401 pressure falls to 0 mmHg.

Following complete deflation, a graph comparing the acoustic sensor 111 magnitude versus the bladder 301/401 pressure is created that captures the period beginning at bladder deflation and ending at complete deflation (step 716).

In an alternate embodiment, a manual inflation bulb 605 is used to inflate the cuff 101 bladder 301/401. The cuff 101 bladder 301/401 is inflated to a supra-systolic BP to completely occlude the brachial artery (step 703).

If a Korotkoff sound is displayed on the GUI 621 in the form of a sound magnitude greater than zero, the user manually re-inflates the cuff 101 bladder 301/401 pressure to a value exceeding the initial inflation pressure (step 705).

The cuff 101 bladder 301/401 pressure is manually reduced at a rate of 1-5 mmHg per second using the bleed valve 607 (step 707). The bleed valve 607 may allow the user to control the deflation rate or may have a fixed deflation rate.

The cuff 101 bladder 301/401 pressure 627 begins to fall at the bleed valve 607 rate. The acoustic sensor 111 acquires sound manifesting in the brachial artery (during partial occlusion), and outputs a voltage magnitude that is compared with the bladder 301/401 pressure 627. A comparison of acoustic sensor 111 sound magnitude versus bladder pressure 627 is captured and stored 617 while the cuff 101 bladder 301/401 pressure falls to 0 mmHg (step 709). The user observes the comparison data and determines the appropriate time to open bleed valve 607 to completely deflate the cuff 101 bladder 301/401 to 0 mmHg (step 713). This may be accomplished by interpreting the final sensor amplitude on the graph, which corresponds to diastolic BP (step 711). The user has the option to not completely open bleed valve 607 during deflation, but this may cause discomfort to the patient.

The cuff 101 bladder 301/401 can be re-inflated either partially or to supra-systolic pressures to plot multiple inflation/deflation cycles on the same graph.

Both the manual and automatic inflation methods incorporate identical signal processing methods following complete bladder 301/401 deflation. Two cursors comprise available inputs allowing the user to manually determine systolic and diastolic BP. The cursors may be dragged anywhere on the graph and display the cuff pressure corresponding to the position of the cursor. The user selects the systolic cursor and positions it over the first acoustic sensor 111 amplitude above baseline. A pressure value is displayed on the GUI 621 to indicate systolic BP (step 721). The user selects the diastolic cursor and positions it over the last acoustic sensor 111 amplitude above baseline (step 722). A pressure value is displayed on the GUI 621 to indicate diastolic BP (step 723).

Pulse rate is a measurement of the number of heart beats in one minute. The processor 613 detects arterial pulsations from sensor 111 and calculates a pulse rate (step 724).

MAP is a measurement of average BP and may be determined from an individual's cardiac output, systemic vascular resistance, and central venous pressure. The true MAP can be obtained by complex methods, but at a typical resting heart rate, it may be estimated from systolic and diastolic BP,

$$MAP ≅ DP + \frac{1}{3} ( SP - DP ) , \qquad ( 2 )$$

where DP is the diastolic BP, SP is the systolic BP, and ⅓ is a weight factor. The processor 613 uses (2) to calculate MAP based on the user's selection of systolic and diastolic BP (step 724).

Other significant clinical data can be calculated by the processor 613 using similar methods.

The acoustic sensor magnitude versus bladder pressure comparison data and calculated values of systolic BP, diastolic BP, pulse rate, MAP, and other data can be saved into storage 617 (step 725). This information can be recalled at a later time using the I/O port 619.

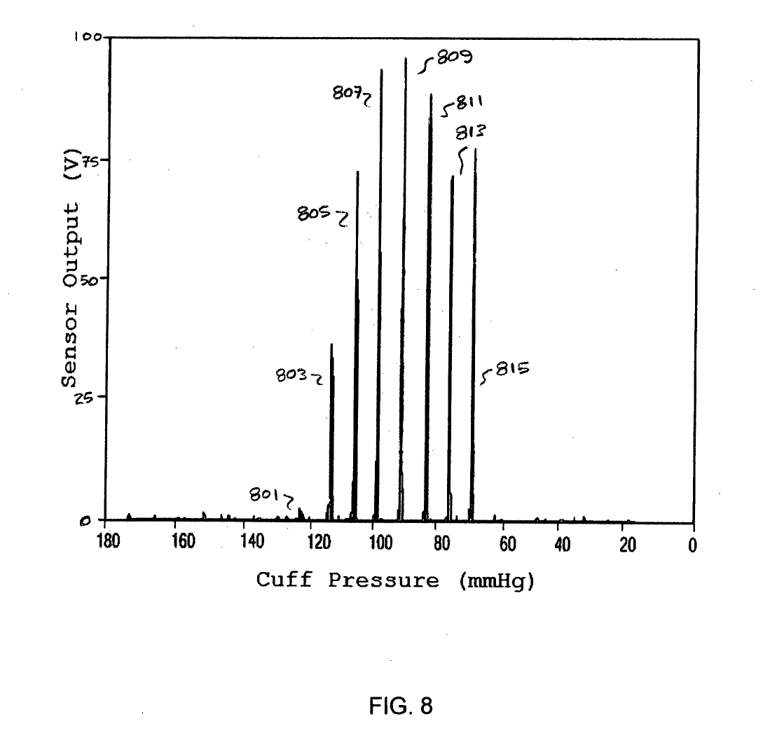

FIG. 8 shows a graph of the acoustic sensor 111 output (ordinate) versus cuff 101 bladder 301/401 pressure 627 (abscissa). The baseline 801 is defined as a nominal voltage measurement. The baseline 801 is present when the acoustic sensor 111 is not transducing arterial pulsations. This occurs when the blood vessel is both completely occluded and completely open. The baseline 801 is also present in between systolic and diastolic BP when the heart is not contracting. Korotkoff sounds are shown by the peak amplitudes 803, 805, 807, 809, 811, 813 and 815 and are only evident when the heart contracts to force blood through the partially-occluded artery. The baseline 801 may comprise some nominal noise artifacts, but it does not interfere with the interpretation of Korotkoff sounds.

The first Korotkoff sound 803 is detected by the acoustic sensor 111 as a substantial voltage response following the baseline 801. The last Korotkoff sound 815 is detected by the acoustic sensor 111 as a substantial voltage response preceding the baseline 801. Korotkoff sounds two through four are detected as peak amplitudes above baseline 801, however, these amplitudes are not considered to be clinically significant. Peak amplitudes 805, 807, 809, 811 and 813 comprise Korotkoff sounds two through four. It is important to note that the peak amplitudes 805, 807, 809, 811 and 813 do not directly coincide with Korotkoff sounds but rather the arterial pulsation of the blood vessel. The number of peak amplitudes is contingent on the deflation rate of the cuff 101 bladder 301/401. If the cuff 101 bladder 301/401 deflates at a rate of 1 mmHg per second, more peak amplitudes will be displayed on the GUI 621 than if the bladder 301/401 deflates at a rate of 5 mmHg.

To determine systolic BP, a systolic cursor (not shown) is positioned over the first Korotkoff sound 803 using the GUI 621. The processor 613 will determine the cuff 101 bladder 301/401 pressure of the systolic cursor based on its horizontal position on the graph. For example, if the first Korotkoff sound 803 were selected with the systolic cursor as in FIG. 8, the systolic BP would be 117 mmHg. The processor 613 accurately determines the systolic pressure and displays the resulting value on the GUI 621.

To determine diastolic BP, a diastolic cursor (not shown) is positioned over the last Korotkoff sound 815 using the GUI 621. The processor 613 will determine the cuff 101 bladder 301/401 pressure of the diastolic cursor based on its horizontal position on the graph. For example, if the last Korotkoff sound 815 were selected with the diastolic cursor as in FIG. 8, the diastolic BP would be 70 mmHg. The processor 613 accurately determines the diastolic pressure and displays the resulting value on the GUI 621.

One or more embodiments of the present invention have been described. Nevertheless, it will be understood that various modifications may be made without departing from the spirit and scope of the invention. Accordingly, other embodiments are within the scope of the following claims.

What is claimed is:

1. A Non-Invasive Blood Pressure (NIBP) monitoring method comprising:
positioning a blood pressure cuff having an inflatable bladder and an acoustic sensor with the acoustic sensor in contact with a surface directly in contact with an artery;
selecting a first predetermined cuff bladder deflation rate;
inflating the cuff bladder;
monitoring acoustic sensor signal values and bladder pressure values during bladder inflation;
determining an approximate systolic Blood Pressure (BP);
inflating the cuff bladder to a pressure exceeding the approximate systolic BP to occlude the artery;
initiating cuff bladder deflation comprising:
bleeding the cuff bladder pressure at the first predetermined cuff bladder deflation rate;
trending the acoustic sensor signal values versus the cuff bladder pressure values;
determining a final acoustic sensor signal value from the trend of acoustic sensor signal values versus cuff bladder pressure values; and
bleeding the cuff bladder pressure at a second predetermined cuff bladder deflation rate until the cuff bladder is completely deflated; and
determining a systolic BP and a diastolic BP from the acoustic sensor signal values versus bladder pressure values trend.

2. The method according to claim 1 wherein the first predetermined cuff bladder deflation rate is 1-5 mmHg per second.

3. The method according to claim 1 wherein the second predetermined cuff bladder deflation rate is a maximum deflation rate.

4. The method according to claim 1 further comprising analyzing all acoustic sensor signal values in the acoustic sensor signal values versus bladder pressure values trend.

5. The method according to claim 1 further comprising displaying the systolic and diastolic BP pressures.

6. The method according to claim 1 further comprising calculating a Mean Arterial Pressure (MAP) from the acoustic sensor signal values versus bladder pressure values trend.

7. The method according to claim 1 further comprising storing the acoustic sensor signal values versus bladder pressure values trend.

8. A Non-Invasive Blood Pressure (NIBP) monitoring cuff comprising:
a cuff comprising:
a top surface and a bottom surface, the top and bottom surfaces configured as mirror images of one another and define an interior aperture;
an inflation tubing barb, wherein the top and bottom surfaces are of a flexible air-tight material and the top, bottom and tubing barb are coupled together and define an internal bladder; and
a fastening means applied to the top and bottom surfaces to secure the cuff when wrapped; and
an acoustic sensor; and
a suspension, wherein the suspension is configured to be positioned in the interior aperture and coupled to the cuff, and the sensor is configured to be coupled to the suspension, wherein the suspension provides acoustic isolation between the cuff and the acoustic sensor, and limits acoustic sensor movement during bladder inflation and deflation after cuff placement.

9. The NIBP monitoring cuff according to claim 8 wherein the fastening means is positioned in matching correspondence on the top and bottom surfaces.

10. The NIBP monitoring cuff according to claim 9 wherein the fastening means is a hook and loop fastener.

11. The NIBP monitoring cuff according to claim 8 wherein the top and bottom surfaces define a length and width such that the cuff may easily wrap around a patient's bicep.

12. The NIBP monitoring cuff according to claim 8 wherein the top surface material may be different from the bottom surface material to define bladder inflation and deflation properties.

13. The NIBP monitoring cuff according to claim 8 wherein the top surface material and bottom surface material are a polymer or copolymer, or a biocompatible, impermeable elastomeric material.

14. The NIBP monitoring cuff according to claim 8 further comprising a one-piece inflatable bladder configured to be located within the internal bladder and coupled to the inflation tubing barb.

15. The NIBP monitoring cuff according to claim 8 wherein the suspension has an aperture in the center configured to allow a flange of the sensor to pass through.

16. The NIBP monitoring cuff according to claim 8 wherein materials to form the suspension include woven polyester and rubber, woven elastic fabrics, formed elastics and latex-free elastics.

17. The NIBP monitoring cuff according to claim 8 wherein the suspension is a corrugated fabric disk.

18. The NIBP monitoring cuff according to claim 8 wherein the acoustic sensor includes transducers, microphones, ceramic bimorphs and piezoelectric materials.

19. The NIBP monitoring cuff according to claim 18 wherein the sensor has a frequency range between 1 and 250 Hz.

20. The NIBP monitoring cuff according to claim 8 wherein the top and bottom surfaces define a length and width, wherein the cuff width is 0.4 times a predetermined circumference defined by the length and the length of the bladder is 0.8 times the length.
